(19) United States
(12) Patent Application Publication
(10) Pub. No.: US 2025/0197641 A1
(43) Pub. Date: Jun. 19, 2025

(54) OPTICAL ABSORBENT

(71) Applicant: LMS CO., LTD., Pyeongtaek-si (KR)

(72) Inventors: Joon Ho JUNG, Pyeongtaek-si (KR); Hee Kyeong KIM, Pyeongtaek-si (KR)

(21) Appl. No.: 18/954,922

(22) Filed: Nov. 21, 2024

(30) Foreign Application Priority Data
Dec. 14, 2023 (KR) 10-2023-0181831

(51) Int. Cl. C09B 23/08; C07F 7/10; C08K 5/544; C09B 23/01; C09D 7/63; C09D 183/04; G01J 1/04

(52) U.S. Cl. CPC C09B 23/083; C07F 7/10; C09B 23/0066; C09D 7/63; C09D 183/04; G01J 1/0422; C08K 5/5477

(57) ABSTRACT

Provided are absorbents and their uses. The absorbent is an organic absorbent. It exhibits excellent compatibility or solubility with various solvents and resin components and has excellent heat resistance. Thus, its optical properties can be stably maintained even when it is maintained under high temperature or high temperature/high humidity conditions. By applying the absorbent, an absorption membrane that obtains desired optical properties can be provided. The specification also provides the use of the absorbent or an absorption membrane.

### CROSS-REFERENCE TO RELATED APPLICATION(S)

This application is based on and claims priority under 35 U.S.C. 119 to Korean Patent Application No. 10-2023-0181831, filed on Dec. 14, 2023, in the Korean Intellectual Property Office, the disclosure of which is herein incorporated by reference in its entirety.

### TECHNICAL FIELD

This specification relates to optical absorbents and their uses.

### BACKGROUND

An optical absorbent, for example, an absorbent capable of absorbing light in the infrared region can be applied to various applications. Because an image capturing device or an infrared sensor using a CCD (Charge-Coupled Device) or a CMOS (Complementary Metal-Oxide-Semiconductor) image sensor, for example, includes a silicon photodiode having sensitivity to the near-infrared region, the optical absorbent may be used for them.

There are various ways to apply these absorbents, but a method using a coating solution that mixes an absorbent dissolved in a solvent and a resin component is usually applied.

Therefore, the absorbent must exhibit excellent solubility or compatibility with both the solvent and the resin component.

If the solubility or compatibility of the absorbent with the solvent or resin component is poor, the desired spectral characteristics cannot be obtained for an absorption membrane to which the absorbent is applied, or optical characteristics are deteriorated due to the phenomenon of precipitation of the absorbent within the absorption membrane. Consequently, it is a difficult task to obtain an absorbent that simultaneously exhibits excellent solubility or compatibility with various types of solvents and resin components.

The absorbents can be divided into inorganic absorbents and organic absorbents. The organic absorbents are easy to be applied and can easily control the wavelength of light to be absorbed, so they can efficiently reduce the transmittance of the desired light.

However, since the organic absorbents have poorer heat resistance than inorganic absorbents, the optical properties of the absorbent or the film containing the absorbent may deteriorate after exposure to high temperature conditions or high temperature and high humidity conditions.

### SUMMARY

The present specification discloses absorbents and their uses.

The object of the present specification is to disclose an organic absorbent that exhibits excellent compatibility or solubility with various solvents and resin components.

Another object of the present specification is to disclose an organic absorbent that has excellent heat resistance and can stably maintain optical properties even when maintained under high temperature conditions or high temperature and high humidity conditions.

Yet another object of the present specification is to disclose an optical filter that can secure desired optical properties by applying the absorbent.

According to an embodiment of the invention, there is provided that an absorbent comprising a cation represented by Formula 1:

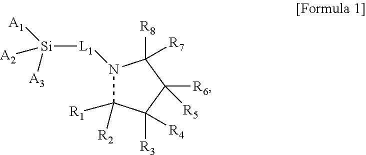

where A₁ to A₃ are each independently hydrogen, halogen, an alkyl group, an alkenyl group, an alkynyl group, an alkoxy group, an aryl group, or an arylalkyl group; L₁ is -U₁-T₁-U₂-T₂-U₃- where T₁ and T₂ are each independently an oxygen atom or absent and U₁ to U₃ are each independently an alkylene group, an alkylidene group, an alkenylene group, or an alkynylene group or absent; R₁ and R₂ form an absorption edge; R₃ to R₆ or R₅ to R₈ among R₃ to R₈ form an absorption edge; and a substituent not forming the absorption edge among R₃ to R₈ are each independently hydrogen, halogen, a hydroxyl group, a cyano group, a nitro group, a carboxyl group, an alkyl group, an alkoxy group, an aryl group, an arylalkyl group, an alkylcarbonylamino group, an arylalkylcarbonylamino group, a haloalkylsulfonylamino group, an alkylsulfonylamino group, an arylalkylsulfonylamino group, or an amino group; and a dotted line in Formula 1 is a single bond or a double bond where R₂ is absent if the dotted line is the double bond; and a cationic site is formed on a nitrogen atom connected to the dotted line if the dotted line is the double bond.

In an embodiment, A₁ to A₃ are each independently the alkyl group, the alkynyl group, the alkenyl group, or the alkoxy group in Formula 1 for the absorbent.

In an embodiment, the absorbent satisfies one of Conditions 1 to 3:
Condition 1: T₁, T₂, U₁ and U₃ are absent and U₂ is an the alkylene group, the alkylidene group, the alkenylene group or the alkynylene group in Formula 1;
Condition 2: U₁ and U₃ are absent; one of T₁ and T₂ is oxygen and the other is absent; and U₂ is the alkylene group, the alkylidene group, the alkenylene group, or the alkynylene group in Formula 1; and
Condition 3: U₃ and T₂ are absent; T₁ is the oxygen atom, and U₁ and U₂ are each independently the alkylene group; the alkylidene group, the alkenylene group, or the alkynylene group.

In an embodiment, the substituent not forming the absorption edge among R₃ to R₈ is each independently hydrogen, halogen, the hydroxy group, the cyano group, the nitro group, the carboxyl group, the alkyl group, the alkoxy group, the alkylsulfonylamino group, or the amino group for the absorbent.

In an embodiment, the cation is represented by Formula 2 and Formula 3:

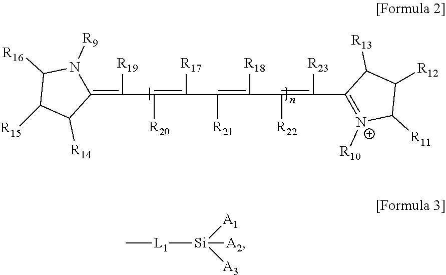

where R₉ and R₁₀ are each independently hydrogen, an alkyl group, or a substituent of Formula 3 where at least one of R₉ and R₁₀ is the substituent of Formula 3; R₁₁ and R₁₂ or R₁₂ and R₁₃ among R₁₁ to R₁₃ form an absorption edge together; R₁₄ and R₁₅ or R₁₅ and R₁₆ among R₁₄ to R₁₆ form an absorption edge together; a substituent not forming the absorption edge among R₁₄ to R₁₆ is each independently hydrogen, halogen, a hydroxyl group, a cyano group, a nitro group, a carboxyl group, an alkyl group, an alkoxy group, an aryl group, an arylalkyl group, an alkylcarbonylamino group, an arylalkylcarbonylamino group, a haloalkylsulfonylamino group, an alkylsulfonylamino group, an arylalkylsulfonylamino group, or an amino group; R₁₇ and R₁₈ are each independently hydrogen, halogen, or an alkyl group or are connected to each other to form a ring structure; R₁₉ to R₂₃ are each independently is hydrogen, halogen or an alkyl group; and n is a number greater than or equal to 1 in Formula 2; and L₁ is -U₁-T₁-U₂-T₂-U₃- where T₁ and T₂ are each independently an oxygen atom or absent and U₁ to U₃ are each independently an alkylene group, an alkylidene group, an alkenylene group, an alkynylene group or absent and L₁ is connected to a nitrogen atom in Formula 2; and A₁ to A₃ are each independently hydrogen, halogen, an alkyl group, an alkenyl group, an alkynyl group, an alkoxy group, an aryl group, or an arylalkyl group in Formula 3.

In an embodiment, R₉ and R₁₀ in Formula 2 are the substituents in Formula 3 for the absorbent.

In an embodiment, the absorbent exhibits an absorption maximum within a wavelength range of 600 nm to 950 nm.

In an embodiment, 5% thermal decomposition temperature of the absorbent is 190° C. or higher.

According to another embodiment of the present invention, a composition is provided to comprise a resin component and the absorbent.

In another embodiment, the resin component includes at least one or more selected from a group consisting of cyclic olefin (COP) based resin, polyester resin, polyarylate resin, polysulfone resin, polyether sulfone resin, polyparaphenylene resin, and polyarylene ether phosphine oxide resin, polyimide resin, polyetherimide resin, polyamidoimide resin, acrylic resin, polycarbonate resin, polyethylene naphthalate resin, and silicone resin for the composition.

In another embodiment, the composition further comprises a solvent.

According to yet another embodiment, an absorption membrane is provided to comprise a resin component and the absorbent.

In yet another embodiment, the resin component includes at least one or more selected from a group consisting of cyclic olefin (COP) based resin, polyester resin, polyarylate resin, polysulfone resin, polyether sulfone resin, polyparaphenylene resin, and polyarylene ether phosphine oxide resin, polyimide resin, polyetherimide resin, polyamidoimide resin, acrylic resin, polycarbonate resin, polyethylene naphthalate resin, and silicone resin for the absorption membrane.

In yet another embodiment, the absorption membrane exhibits an absorption maximum within a wavelength range of 600 nm to 950 nm.

In yet another embodiment, an absolute value of ΔA in Equation 1 is 10% or less:

$$Δ A = 100 \times ( {A}_{f} - {A}_{i} ) / {A}_{i} , \qquad [ Equation 1 ]$$

where A_{f} is a transmittance at an absorption maximum wavelength of the absorption membrane after the absorption membrane is maintained at 85° C. under 85% relative humidity condition for 120 hours and Aᵢ is a transmittance at an absorption maximum wavelength of the absorption membrane before the absorption membrane is maintained at 85° C. under 85% relative humidity condition for 120 hours in Equation 1 and the absorption maximum wavelength exists within a wavelength range of 600 nm to 950 nm for the absorption membrane.

In yet another embodiment, an absolute value of Δλ in Equation 2 is 10% or less:

$$Δ λ = 100 \times ( {λ}_{f} - {λ}_{i} ) / {λ}_{i} , \qquad [ Equation 2 ]$$

where λ_{f} is an absorption maximum wavelength of the absorption membrane after the absorption membrane is maintained at 85° C. under 85% relative humidity condition for 120 hours and λᵢ is an absorption maximum wavelength of the absorption membrane before the absorption membrane is maintained at 85° C. under 85% relative humidity condition for 120 hours in Equation 2; and the absorption maximum wavelength exists within a wavelength range of 600 nm to 950 nm for the absorption membrane.

According to yet another embodiment, an optical filter is provided to comprise a substrate and the absorption membrane formed on one or both sides of the substrate.

According to yet another embodiment, an image capturing device is provided to comprise the optical filter of claim 17.

According to yet another embodiment, an infrared sensor is provided to comprise the absorption membrane.

### BRIEF DESCRIPTION OF THE DRAWINGS

FIGS. 1 to 3 are drawings showing exemplary structures of optical filters of the present invention.

FIGS. 4 to 6 are spectra showing the light absorption characteristics of absorption membranes containing absorbents of Embodiments 1 to 3 before and after high-temperature/high-humidity evaluation.

FIGS. 7 to 9 are spectra showing the light absorption characteristics of absorption membranes containing absorbents of Comparative Examples 1 to 3 before and after high-temperature/high-humidity evaluation.

### DETAILED DESCRIPTION

For those physical properties mentioned in the present invention where the result of measuring temperature may affect, it is measured at room temperature unless otherwise specified. The term “room temperature” used in the present invention refers to a natural temperature that is not heated or not reduced, for example, it means any temperature within the range of 10° C. to 30° C., a temperature of about 23° C. or about 25° C. In addition, in the present specification, the unit of temperature is Celsius (° C.) unless otherwise specified.

Among the physical properties mentioned in the present specification, in case where the measured pressure affects the result, the physical property is a physical property measured at atmospheric pressure unless specifically mentioned. The term “atmospheric pressure” is a natural pressure that is not pressurized or depressurized. It usually indicates that about 1 atmosphere of atmospheric pressure having the value of about 740 mmHg to 780 mmHg.

Among the physical properties mentioned in the present specification, in cases where humidity affects the results, the relevant physical properties are those measured at standard humidity unless otherwise specified. Humidity in a standard state means any humidity in the range of 40% to 60% relative humidity, for example, about 40% or 60% relative humidity.

In the case where the optical characteristic (for example, refractive index) mentioned in the present specification is a characteristic that varies depending on the wavelength, the optical characteristic is a characteristic for light with a wavelength of 520 nm unless specifically specified otherwise.

In the present specification, the terms “transmittance,” “reflectance,” or “absorption rate” refer to the actual transmittance (actual transmittance), actual reflectance (actual reflectance), or actual absorption (actual absorption rate) confirmed within a specific wavelength or wavelength range of a predetermined region unless specifically defined otherwise. In the present specification, the terms transmittance, reflectance, or absorption rate refer to transmittance, reflectance, or absorption with respect to an incident angle of 0° unless specifically defined otherwise.

In the present specification, the term “average transmittance” is the result of measuring the transmittance at each wavelength while increasing the wavelength by 1 nm starting from the shortest wavelength within a certain wavelength range, and then calculating the arithmetic average of the measured transmittances unless specifically defined otherwise. For example, the average transmittance within the wavelength range of 350 nm to 360 nm may be the arithmetic mean of the transmittance measured at the wavelength of 350 nm, 351 nm, 352 nm, 353 nm, 354 nm, 355 nm, 356 nm, 357 nm, 358 nm, 359 nm, and 360 nm.

In the present specification, the term “maximum transmittance” is the maximum transmittance when the transmittance of each wavelength is measured while increasing the wavelength by 1 nm starting from the shortest wavelength within a certain wavelength range. For example, the maximum transmittance within the wavelength range of 350 nm to 360 nm may be the highest transmittance measured at the wavelength of 350 nm, 351 nm, 352 nm, 353 nm, 354 nm, 355 nm, 356 nm, 357 nm, 358 nm, 359 nm, and 360 nm.

In present specification, the term “average reflectance” is the result of measuring the reflectance at each wavelength while increasing the wavelength by 1 nm starting from the shortest wavelength within a predetermined wavelength range, and then calculating the arithmetic average of the measured reflectance unless specifically defined otherwise. For example, the average reflectance within the wavelength range of 350 nm to 360 nm may be the arithmetic mean of the reflectance measured at the wavelength of 350 nm, 351 nm, 352 nm, 353 nm, 354 nm, 355 nm, 356 nm, 357 nm, 358 nm, 359 nm, and 360 nm.

In the present specification, the term “maximum reflectance” is the maximum reflectance when the reflectance of each wavelength is measured while increasing the wavelength by 1 nm starting from the shortest wavelength within a certain wavelength range. For example, the maximum reflectance within the wavelength range of 350 nm to 360 nm may be the highest reflectance measured at any given wavelength of 350 nm, 351 nm, 352 nm, 353 nm, 354 nm, 355 nm, 356 nm, 357 nm, 358 nm, 359 nm, and 360 nm.

In the present specification, the term “average absorption rate” is the result of measuring the absorption rate at each wavelength while increasing the wavelength by 1 nm starting from the shortest wavelength within a predetermined wavelength range, and then calculating the arithmetic average of the measured absorption rates unless specifically defined otherwise. For example, the average absorption rate within the wavelength range of 350 nm to 360 nm may be the arithmetic mean of the absorption rate measured at the wavelength of 350 nm, 351 nm, 352 nm, 353 nm, 354 nm, 355 nm, 356 nm, 357 nm, 358 nm, 359 nm, and 360 nm.

In the present specification, the term “maximum absorption rate” is the maximum absorption rate when measuring the absorption rate at each wavelength while increasing the wavelength by 1 nm starting from the shortest wavelength within a certain wavelength range. For example, the maximum absorption rate within the wavelength range of 350 nm to 360 nm may be the highest absorption rate among those measured at wavelength of 350 nm, 351 nm, 352 nm, 353 nm, 354 nm, 355 nm, 356 nm, 357 nm, 358 nm, 359 nm and 360 nm.

In the present specification, the “incident angle” is an angle measured with respect to the normal line of the surface to be evaluated. For example, “the transmittance at an incident angle of 0° of an optical filter” means the transmittance of light incident in a direction substantially parallel to the normal line of the surface of the optical filter. In addition, for example, an incident angle of 40° is a value for incident light forming an angle of substantially 400 in a clockwise or counterclockwise direction with the normal. This definition of the incident angle applies equally to other characteristics such as transmittance.

In the present specification, the term “alkyl group” means an alkyl group having 1 to 20 carbon numbers, 1 to 16 carbon numbers, 1 to 12 carbon numbers, 1 to 8 carbon numbers, or 1 to 4 carbon numbers. The alkyl group can be straight-chain, branched-chain or cyclic. The alkyl group may optionally be substituted with one or more substituents. These contents are applied to all alkyl groups mentioned in the specification unless otherwise specified.

In the present specification, the term “alkoxy group” means an alkoxy group having 1 to 20 carbon numbers, 1 to 16 carbon numbers, 1 to 12 carbon numbers, 1 to 8 carbon numbers, or 1 to 4 carbon numbers. The alkoxy group can be straight-chain, branched-chain or cyclic.

The alkoxy group may be optionally substituted with one or more substituents. These contents are applied to all alkoxy groups mentioned in the specification unless otherwise specified.

In the present specification, the term “alkenyl group” means an alkenyl group having 2 to 20 carbon numbers, 2 to 16 carbon numbers, 2 to 12 carbon numbers, 2 to 8 carbon numbers, or 2 to 4 carbon numbers. The alkyl group can be straight-chain, branched-chain or cyclic.

The alkenyl group may be optionally substituted with one or more substituents. These contents apply to all alkenyl groups mentioned in the specification unless otherwise specified.

In the present specification, the term “alkenyl group” means an alkenyl group having 2 to 20 carbon numbers, 2 to 16 carbon numbers, 2 to 12 carbon numbers, 2 to 8 carbon numbers, or 2 to 4 carbon numbers. The alkyl group can be straight-chain, branched-chain or cyclic.

The alkenyl group may be optionally substituted with one or more substituents. These contents apply to all alkenyl groups mentioned in the specification unless otherwise specified.

In the specification, the term “aryl group” means a monovalent moiety derived from an aromatic hydrocarbon, and the aryl group may be an aryl group having 6 to 36 carbon numbers, 6 to 30 carbon numbers, 6 to 24 carbon numbers, 6 to 18 carbon numbers, or 6 to 12 carbon numbers. It may be, for example, a phenyl group, a tolyl group, a xylyl group, or a naphthyl group. The aryl group may optionally be substituted by one or more substituents. These contents are applied to all aryl groups mentioned in the specification unless otherwise specified.

In the present specification, the term “arylalkyl group” means an alkyl group substituted with at least one aryl group, and in this case, specific types of the aryl groups are as described above. This applies to all arylalkyl groups mentioned in this specification, unless specifically otherwise stated.

In the present specification, the term “alkylidene group” means a divalent functional group in which two hydrogens have been removed from an alkane and it also means a functional group where the hydrogen atom has been removed from one carbon of the alkane. Such an alkylidene group may be an alkylidene group having 1 to 20 carbon numbers, 1 to 16 carbon numbers, 1 to 12 carbon numbers, 1 to 8 carbon numbers, or 1 to 4 carbon numbers. The alkylidene group may be straight chain, branched chain, or cyclic. The alkylidene group may be arbitrarily substituted with one or more substituents. This applies to all alkylidene groups mentioned in this specification unless specifically stated otherwise.

In the present specification, the term “alkylene group” refers to a divalent functional group in which two hydrogen atoms are removed from an alkane and it further means that one hydrogen atom is removed from each of two different carbons of the alkane. Such an alkylene group may be an alkylene group having 2 to 20 carbon numbers, 2 to 16 carbon numbers, 2 to 12 carbon numbers, 2 to 8 carbon numbers, or 2 to 4 carbon numbers. The alkylene group may be straight chain, branched chain, or cyclic. The alkylene group may be arbitrarily substituted with one or more substituents. This applies to all alkylene groups mentioned in the specification unless specifically stated otherwise.

In the present specification, the term “alkenylene group” may be an alkenylene group having 2 to 20 carbon numbers, 2 to 16 carbon numbers, 2 to 12 carbon numbers, 2 to 8 carbon numbers, or 2 to 4 carbon numbers. The alkenylene group may be straight chain, branched chain, or cyclic. The alkenylene group may be arbitrarily substituted with one or more substituents. This applies to all alkenylene groups mentioned in the specification unless specifically stated otherwise.

In the present specification, the term “alkynylene group” may be an alkynylene group having 2 to 20 carbon numbers, 2 to 16 carbon numbers, 2 to 12 carbon numbers, 2 to 8 carbon numbers, or 2 to 4 carbon numbers. The alkynylene group may be straight chain, branched chain, or cyclic. The alkynylene group may be arbitrarily substituted with one or more substituents. This applies to all alkynylene groups mentioned in the specification unless specifically stated otherwise.

The present specification discloses an absorbent. The term “absorbent” refers to a chemical compound capable of absorbing light in a certain wavelength range.

The absorbent may be a chemical compound containing a cation represented by Formula 1 below or a cation containing a portion represented by Formula 1 below. The absorbent can exhibit excellent heat resistance, desired optical properties, and excellent compatibility with resin components and/or solvents by the silyl group connected to the nitrogen atom in the following Formula 1.

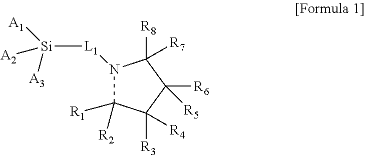

In Formula 1, A₁ to A₃ may each independently be hydrogen, halogen, an alkyl group, an alkenyl group, an alkynyl group, an alkoxy group, an aryl group, or an arylalkyl group. In Formula 1, L₁ may be a divalent functional group represented by “-U₁-T₁-U₂-T₂-U₃-.” In the divalent functional group, T₁ and T₂ may each independently be an oxygen atom or may not exist, and U₁ to U₃ may each independently be an alkylene group, an alkylidene group, an alkenylene group, or an alkynylene group or may not exist. In the above, the absence of a certain symbol means that the atoms on both sides of the corresponding symbol are directly connected. For example, in the above, when elements other than T₁ exist but T₁ does not exist, the divalent functional group may be expressed as “-U₁-U₂-T₂-U₃-.”

R₁ and R₂ in Formula 1 may form an absorption edge. R₃ to R₆ or R₅ to R₈ among R₃ to R₈ may also form an absorption edge. Forming an absorption edge in the above means that the absorbent has a structure capable of absorbing light of a desired wavelength as a whole due to the structure formed by the substituent or the structure formed at the position where the substituent exists. The absorption stage will be described later. Other substituents that do not form the absorption edge among R₃ to R₈ in Formula 1 may be each independently hydrogen, halogen, a hydroxy group, a cyano group, a nitro group, a carboxyl group, an alkyl group, an alkoxy group, an aryl group, an arylalkyl group, an alkyl carbonylamino group, an arylalkylcarbonylamino group, an alkylsulfonylamino group, a haloalkylsulfonylamino group, an arylalkylsulfonylamino group, or an amino group.

In Formula 1, the dotted line indicates that the dotted line is a single bond of nitrogen and carbon or a double bond of nitrogen and carbon. When the dotted line in Formula 1 is a double bond of nitrogen and carbon, R₂ does not exist among R₁ and R₂ and R₁ forms the absorption edge. Additionally, when the dotted line is a double bond of nitrogen and carbon, the nitrogen atom in Formula 1 becomes a cation site.

The absorption edge refers to a portion where the absorbent has a structure that allows it to absorb light of a desired wavelength as a whole. For example, the absorption edge may be a frame or structure having a so-called resonance structure and/or conjugated bond.

Light absorption by an absorbent—especially an organic absorbent—is known to be caused by the energy difference (ΔE) between the ground state and the excited state. This difference is explained by the energy difference of HOMO (Highest Unoccupied Molecular Orbital) and LUMO (Lowest Unoccupied Molecular Orbital).

In general, organic absorbents include a resonance structure and/or conjugate bond as an absorption edge that can exhibit a light absorption effect. Accordingly, R₁ and R₂ (or R₁ when the dotted line in Formula 1 is a double bond) and R₃ to R₆ or R₅ to R₈ are connected to a frame that allows the absorbent to exhibit desired light absorption characteristics as a whole including the resonance structure and/or conjugate bond, or together form such a frame.

The absorption edge or the frame may not be particularly limited to a specific type. As is known, the resonance effect refers to the interaction between a lone electron pair of a molecule and an adjacent π bonded electron pair. The substituents or frames that cause such resonance effect are known in public. In addition, a conjugated bond is a system composed of two or more double bonds with one single bond in-between. It is known that as the conjugated bond increases, the energy difference decreases and the absorption band shifts to the long wavelength range.

For example, the absorption edge may be a frame or structure that allows the chemical compound of the present invention to exhibit an absorption maximum within a wavelength range of 600 nm to 950 nm. In other words, the chemical compound may exhibit a maximum absorption wavelength within the range of 600 nm to 950 nm. The lower limit of the absorption maximum wavelength may be about 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, or 810 nm and the upper limit may be about 950 nm, 940 nm, 930 nm, 920 nm, 910 nm, 900 nm, 890 nm, 880 nm, 870 nm, 860 nm, 850 nm, 840 nm, 830 nm, 820 nm, 810 nm, 800 nm, 790 nm, 780 nm, or 770 nm. The absorption maximum wavelength may be within a range that is equal to or exceeding any one of the lower limits described above; within a range that is equal to or below any one of the above-described upper limits; or within a range that is equal to or exceeding any one of the above-described lower limits and equal to or below any one of the above-described upper limits.

As mentioned above, the resonance structure and conjugation bond are determined by the energy difference (ΔE) between the ground state and excited state of the chemical compound or the difference between HOMO (Highest Unoccupied Molecular Orbital) and LUMO (Lowest Unoccupied Molecular Orbital). Because the energy difference is determined and the maximum absorption wavelength is determined by this energy difference, the structure of the absorption edge can be determined so that the chemical compound can have the maximum absorption wavelength in the range described above.

In Formula 1, an alkyl group, an alkenyl group, an alkynyl group, an alkoxy group, an aryl group, an arylalkyl group, an alkylene group, an alkylidene group, an alkenyl group, an alkynyl group, an alkylcarbonylamino group, an arylalkylcarbonylamino group, a haloalkylsulfonylamino group, an arylalkylsulfonylamino group, an alkylsulfonylamino group, and an amino group may be arbitrarily substituted with one or more substituents. In this case, the substituted substituents may include halogen such as fluorine or chlorine, an alkyl group, an alkenyl group, an alkynyl group, and an alkoxy group, an aryl group, an arylalkyl group, a hydroxy group, a cyano group, a nitro group, a carboxyl group, an alkylcarbonylamino group, an arylalkylcarbonylamino group, an arylalkylsulfonylamino group, an alkylsulfonylamino group, or an amino group, etc., but are not limited to.

In Formula 1, A₁ to A₃ may each independently be an alkyl group, an alkynyl group, an alkenyl group, or an alkoxy group or may be an alkyl group in appropriate examples. But, they are not limited to.

In Formula 1, the divalent functional group represented by “-U₁-T₁-U₂-T₂-U₃-” may satisfy any one of the following Conditions 1 to 3 in appropriate examples. For example, the divalent functional group may be a functional group where T₁, T₂, U₁, and U₃ do not exist and U₂ is an alkylene group, an alkylidene group, an alkenylene group or an alkynylene group (Condition 1). In this case, the divalent functional group may be represented as “-U₂-.”

In another example, the divalent functional group may be a functional group where U₁ and U₃ do not exist, any one of T₁ or T₂ is oxygen and the other does not exist, and U₂ is an alkylene group, an alkylidene group, an alkenyl group, or an alkynylene group (Condition 2). In this case, the divalent functional group becomes a functional group represented by “—O—U₂-” or “-U₂-O—.”

In another example, the divalent functional group may be a functional group where U₃ and T₂ do not exist, T₁ is oxygen atom, and U₁ and U₂ are each independently an alkylene group, an alkylidene group, an alkenylene group, or an alkynylene group. (Condition 3). In this case, the functional group becomes a functional group represented by “-U₁-OU₂-.”

Among R₃ to R₈ of Formula 1, the substituents that do not form the absorption edge or are not connected to the absorption edge are, in appropriate examples, each independently hydrogen, halogen, a hydroxy group, a cyano group, a nitro group, a carboxyl group, an alkyl group, and an alkoxy group, an alkylsulfonylamino group, or an amino group or hydrogen, an alkyl group, an alkylsulfonylamino group, or an amino group. However, they are not limited to.

The chemical compound (absorbent) may have an appropriate level of molar mass. For example, the lower limit of the molar mass may be about 400 g/mol, 450 g/mol, 500 g/mol, 550 g/mol, 600 g/mol, 650 g/mol, 700 g/mol or 750 g/mol. The upper limit of the molar mass may be about 2,000 g/mol, 1,900 g/mol, 1,800 g/mol, 1,700 g/mol, 1,600 g/mol, 1,500 g/mol, 1,400 g/mol, 1,300 g/mol, 1,200 g/mol, 1,100 g/mol, 1,000 g/mol, 950 g/mol, 900 g/mol, 850 g/mol, or 800 g/mol. The molar mass may be within a range that is at equal to or exceeding any one of the lower limits described above; within a range that is equal to or below any one of the above-described upper limits; or within a range that is equal to or exceeding any one of the above-described lower limits and equal to or below any one of the above-described upper limits.

The absorbent may have excellent heat resistance. For example, the absorbent may have a 5% thermal decomposition temperature (“Td 5%”) within a predetermined range. For example, the lower limit of Td 5% of the absorbent may be around 190° C., 200° C., 210° C., 220° C., or 230° C. and the upper limit may be 400° C., 380° C., 360° C., 340° C., 320° C., 300° C., 280° C., 275° C., 270° C., 265° C., 260° C., 255° C., 240° C., 235° C., 230° C., 225° C., 220° C., or 215° C. The Td 5% may be within a range that is equal to or exceeding any one of the lower limits described above; within a range that is equal to or below any one of the above-described upper limits; or within a range that is equal to or exceeding any one of the above-described lower limits and equal to or below any one of the above-described upper limits.

The Td 5% is a value obtained through TGA (Thermogravimetric Analysis) analysis. It is the value (Td 5%) at a weight loss of 5% confirmed under the conditions of a temperature range of 25° C. to 800° C., a temperature increase rate of 10° C./min, and a nitrogen (N₂) atmosphere of 60 cm³/min.

In one example, the absorbent may be a chemical compound represented by the following Formula 2.

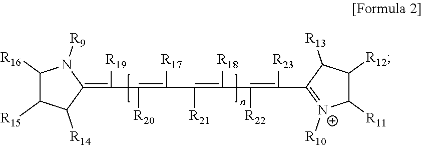

In the structure of Formula 2, the conjugated structure formed between nitrogen atoms may be a frame of the absorption edge formed by R₁ and R₂ of Formula 1 above.

In Formula 2, R₉ and R₁₀ may each independently be hydrogen, an alkyl group, or a substituent of Formula 3 below. In one example, at least one of R₉ and R₁₀ of Formula 2 may be a substituent of Formula 3 below and both R₉ and R₁₀ may be substituents of Formula 3 below.

In Formula 2, among R₁₁ to R₁₃, R₁₁ and R₁₂ may form the absorption edge together, or R₁₂ and R₁₃ may form the absorption edge together. In Formula 2, among R₁₄ to R₁₆, R₁₄ and R₁₅ may form the absorption edge together or R₁₅ and R₁₆ may form the absorption edge together. The specific types of substituents among R₁₁ to R₁₆ in Formula 2 that do not form an absorption edge are the same as those of the substituents among R₃ to R₈ in Formula 1 that do not form an absorption edge.

In Formula 2, “n” is an arbitrary number. The lower limit of “n” may be, for example, about 0 or 1 and the upper limit may be about 10, 9, 8, 7, 6, 5, 4, 3, 2, or 1. The “n” is within a range that is equal to or exceeding any one of the lower limits described above; within a range that is equal to or below any one of the above-described upper limits; or within a range that is equal to or exceeding any one of the above-described lower limits and equal to or below any one of the above-described upper limits.

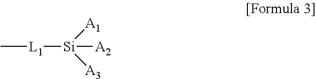

In Formula 3, L₁ is connected to the nitrogen atom in Formula 2. The specific type of L₁ in Formula 3 is the same as that of L₁ in Formula 1. The specific types of A₁ to A₃ in Formula 3 are also the same as A₁ to A₃ in Formula 1, respectively.

The present specification also discloses an absorbent composition comprising the above absorbent. The term “absorbent composition” may refer to a mixture containing an absorbent and other component or a mixture containing two or more types of absorbents.

Such the absorbent composition basically contains the absorbent of Formula 1 or 2 and may additionally contain other necessary components. For example, the composition may further include a resin component that acts as a binder. There is no particular limitation on the type of resin component applied in this case. Known resin components used to form an absorption membrane, for example, a near-infrared absorption membrane can be applied. In the present application, the absorbent component may exhibit appropriate compatibility or solubility with the various known resin components.

Examples of resin components may include one or more kinds selected from cyclic olefin (COP) based resin, polyarylate resin, polyester resin, polysulfone resin, polyether sulfone resin, polyparaphenylene resin, polyarylene ether phosphine oxide resin, polyimide resin, polyetherimide resin, polyamidoimide resin, acrylic resin, polycarbonate resin, polyethylene naphthalate resin, or silicone resin, or various other organic resins or organic-inorganic hybrid resins, but it is not limited to. Although not particularly limited, the compound of the present specification can be mixed with cyclic olefin (COP) based resin among the resin components that serve as a known binder to form a resin film showing excellent performance. Therefore, in one example, the resin component may be cyclic olefin (COP) based resin.

When the resin component is applied, there is no particular limitation to its ratio. For example, the resin component may be present such that the weight ratio of the chemical compound to 100 parts by weight of the resin component ranges from 0.001 parts by weight to 10 parts by weight. The lower limits of the weight ratio of the chemical compound to 100 parts by weight of the resin component may be, in other examples, about 0.001 part by weight, 0.005 part by weight, 0.01 part by weight, 0.05 part by weight, 0.1 part by weight, 0.5 part by weight, 1 part by weight, 1.1 part by weight, 1.2 parts by weight, 1.3 parts by weight, or 1.4 parts by weight and the upper limit may be about 10 parts by weight, 9 parts by weight, 8 parts by weight, 7 parts by weight, 6 parts by weight, 5 parts by weight, 4 parts by weight, 3 parts by weight, 2 parts by weight, or 1.5 parts by weight. The ratio may be within a range that is equal to and greater than or greater than any one of the lower limits described above and is equal to and less or less than any one of the above-described upper limits.

For example, the absorbent composition may further include a solvent in which the absorbent and/or the resin component is dispersed. There is no particular limitation on the type of solvent applied in this case and any known solvent used to form a resin film, for example, a near-infrared resin film can be applied. In the present specification, the absorbent component may exhibit appropriate compatibility or solubility in various known solvents.

Examples of solvents may include methylene chloride, cyclohexanone, toluene, methyl ethyl ketone, methyl isobutyl ketone, propylene glycol methyl ether acetate, diethylene glycol monoethyl ether 3-methoxy butanol, ethylene glycol monobutyl ether acetate, 4-hydroxy-4-methyl-2-pentanone, gammabutyrolactone, cyclohexanone, pyridone, chloroform, 1,4-dioxane, cyclohexanone, ortho-dichlorobenzene, chlorobenzene, aliphatic alcohols having 2 or more carbon numbers (e.g., isobutyl alcohol, isopropyl alcohol, ethanol, isopropanol, butanol, etc.), butyl acetate, tetrahydrofuran, or xylene, but are not limited to. When a solvent is applied, there is no particular limitation on the ratio, and the ratio can be adjusted within a range that allows appropriate dispersion of the absorbent and/or resin component.

The absorbent composition may include other necessary components in addition to the components described above. For example, a type of absorbent different from the absorbent of Formula 1 or 2, etc.

The present specification also discloses the absorbent composition or use of the absorbent. For example, it may be about an absorption membrane to which the absorbent composition or absorbent is applied.

This absorption membrane may contain at least a resin component and the absorbent.

In this case, the specific type of the resin component and the ratio of the resin component to the absorbent are as described in the absorbent composition section.

The absorption membrane may be a film capable of absorbing light within a wavelength range of a predetermined range. In one example, the absorption membrane may be an infrared absorption membrane or a near-infrared absorption membrane. For example, such an absorption membrane may exhibit absorption characteristics in at least a portion of the wavelength range within the range of about 600 nm to 950 nm.

For example, the absorption membrane may exhibit a maximum absorption wavelength within the range of 600 nm to 950 nm. In another examples, the lower limit of the absorption maximum wavelength may be about 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, or 810 nm. In addition, the upper limit of the maximum absorption wavelength may be about 950 nm, 940 nm, 930 nm, 920 nm, 910 nm, 900 nm, 890 nm, 880 nm, 870 nm, 860 nm, 850 nm, 840 nm, 830 nm, 820 nm. nm, 810 nm, 800 nm, 790 nm, 780 nm, 770 nm, 760 nm, 750 nm, 740 nm, 730 nm, 720 nm, 710 nm, 700 nm, 690 nm, 680 nm, 670 nm, 660 nm, 650 nm, 640 nm, 630 nm, 620 nm, or 610 nm. The absorption maximum wavelength may be within a range of equal to or longer than any one of the above-described lower limits and equal to or lower than any one of the above-described upper limits. Due to these characteristics, the absorption membrane can be applied to devices such as various optical filters and infrared sensors because it can provide excellent optical characteristics such as preventing shift phenomenon depending on the incident angle and physical properties such as excellent heat resistance.

For example, the absorption membrane may have an absolute value of ΔA in Equation 1 below within a predetermined range.

$$Δ A = 100 \times ( {A}_{f} - {A}_{i} ) / {A}_{i} . \qquad [ Equation 1 ]$$

In Equation 1, A_{f} is the transmittance at the absorption maximum wavelength of the resin film after being maintained at 85° C. and 85% relative humidity for 120 hours, and Aᵢ is the transmittance at the absorption maximum wavelength of the resin film before being maintained at 85° C. and 85% relative humidity for 120 hours. The absorption maximum wavelength exists within the wavelength range of 690 nm to 950 nm.

The upper limit of the absolute value of ΔA in Equation 1 may be about 20%, 19%, 18%, 17%, 16%, 15%, 14%, 13%, 12%, 11%, 10%, 9%, 8%, 7%, 6%, 5%, or 4.5%, and the lower limit may be 0%, 0.5%, 1%, 1.5%, 2%, 2.5%, 3%, 3.5%, 4%, 4.5%, 5%, 5.5%, 6%, 6.5% or 7%. The absolute value of ΔA may be within a range that is equal to or below any one of the upper limits described above or within a range that is equal to or above any one of the above-described lower limits and equal to or below any one of the above-described upper limits.

In Equation 1, the upper limits of each of A_{f} and Aᵢ may be about 40%, 35%, 30%, 25%, 20%, 19%, 18%, 17%, 16%, 15%, 14%, 13%, 12%, 11%, 10%, 9%, 8%, or 7%, and the lower limit may be about 0%, 0.5%, 1%, 1.5%, 2%, 2.5%, 3%, 3.5%, 4%, 4.5%, 5%, 5.5%, 6%, 6.5%, 7%, 7.5%, or 8%. Each of A_{f} and Aᵢ may be within a range that is equal to or below any one of the upper limits described above or within a range that is equal to or above any one of the above-described lower limits and equal to or below any one of the above-described upper limits.

The absorption membrane may have an absolute value of Δλ in Equation 2 below of 10% or less.

$$Δ λ = 100 \times ( {λ}_{f} - {λ}_{i} ) / {λ}_{i} . \qquad [ Equation 2 ]$$

In Equation 2, λ_{f} is the absorption maximum wavelength of the resin film after being maintained at 85° C. and 85% relative humidity for 120 hours and λᵢ is the absorption maximum wavelength of the resin film before being maintained at 85° C. and 85% relative humidity for 120 hours. The absorption maximum wavelength exists within the wavelength range of 690 nm to 950 nm.

The upper limit of the absolute value of αλ in Equation 2 may be about 10%, 9%, 8%, 7%, 6%, 5%, 4%, 3%, 2%, 1%, or 0.5% in another examples and the lower limit may be about 0%, 0.5%, or 1%. The absolute value of Δλ may be within the range of equal to and more than any one of the above-described lower limits and equal to and below any one of the above-described upper limits.

In Equation 2, λ_{f} and λᵢ may each be in the range of 600 nm to 950 nm. In another examples, the lower limits of each of λ_{f} and λᵢ may be about 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, or 790 nm. In addition, the upper limits of each of λ_{f} and λᵢ may be about 950 nm, 940 nm, 930 nm, 920 nm, 910 nm, 900 nm, 890 nm, 880 nm, 870 nm, 860 nm, 850 nm, 840 nm, 830 nm, 820 nm, 810 nm, 800 nm, 790 nm, 780 nm, 770 nm, 760 nm, 750 nm, 740 nm, 730 nm, 720 nm, 710 nm, 700 nm, 690 nm, 680 nm, 670 nm, 660 nm, 650 nm, 640 nm, 630 nm, 620 nm, or 610 nm. Each of the above λ_{f} and λᵢ may be within a range equal to and greater than any one of the above-described lower limits and equal to and below any one of the above-described upper limits.

Through the absorption properties, the absorption membrane can be applied to various devices such as optical filters and infrared sensors to efficiently achieve the desired properties. The absorption membrane can be formed in a known manner as long as the absorbent composition or absorbent is applied. For example, the absorbent composition may be coated in an appropriate manner, and if necessary, a curing or drying process may be performed to form the absorption membrane.

There is no particular limitation on the thickness of the absorption membrane and the thickness can be adjusted for considering desired characteristics. In one example, the absorption membrane may have a thickness ranging from approximately 0.5 μm to 20 μm.

The present specification also discloses an optical filter. The optical filter may include a substrate and the absorption membrane formed on one or both sides of the substrate.

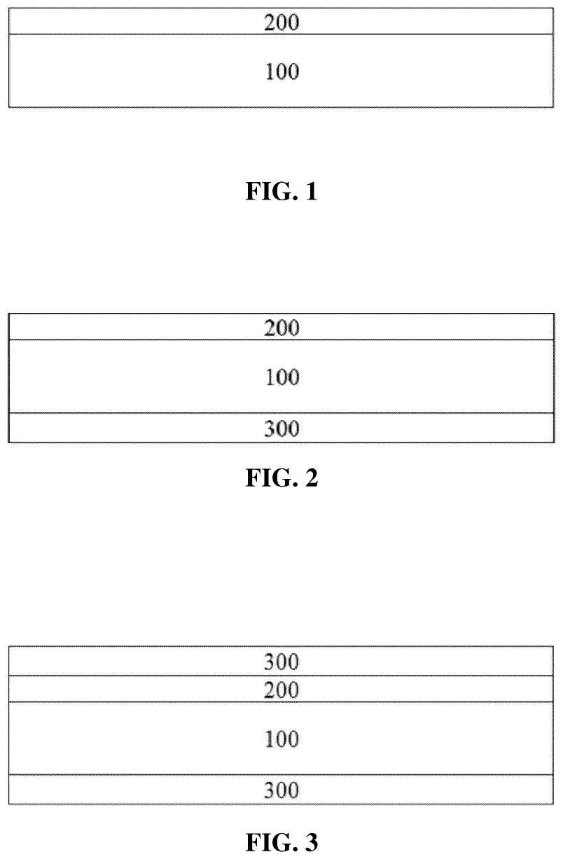

FIG. 1 is an example of the optical filter showing a case where the absorption membrane 200 is formed on one side of the substrate 100. The optical filter of the present specification can exhibit excellent performance by including the above-described resin film. For example, the optical filter can efficiently and accurately block unnecessary infrared light and implement a visible light transmission band with high transmittance.

There is no particular limitation on the type of transparent substrate applied to the optical filter and publicly known transparent substrates for the optical filter can be used. In one example, the substrate may be a so-called infrared absorption substrate. An infrared absorption substrate is a substrate that exhibits absorption characteristics in at least a portion of the near-infrared region. A so-called blue glass, which exhibits the above-mentioned properties by including copper, is a representative example of the infrared absorption substrate. Such the infrared absorption substrate is useful in constructing an optical filter that blocks light in the infrared region, but due to the absorption characteristics, it is disadvantageous in terms of securing high transmittance in the visible light region and is also disadvantageous in terms of durability. In the present specification, an optical filter that efficiently blocks desired light, exhibits high transmittance characteristics in the visible light region, and has excellent durability can be provided by properly selecting an infrared absorption substrate and combining it with the specific resin film.

As for the infrared absorption substrate, a substrate exhibiting an average transmittance of 75% or more within the range of 425 nm to 560 nm can be used. In another examples, the average transmittance may be within the range of about 77% or more, 79% or more, 81% or more, 83% or more, 85% or more, 87% or more, or 89% or more and/or 98% or less, 96% or less, 94% or less, 92% or less, or 90% or less.

As for the infrared absorption substrate, a substrate showing a maximum transmittance of 80% or more within the range of 425 nm to 560 nm can be used. In another examples, the maximum transmittance may be within a range of about 82% or more, 84% or more, 86% or more, 88% or more, or 90% or more and/or 100% or less, 98% or less, 96% or less, 94% or less, 92% or less, or 90% or less.

As for the infrared absorption substrate, a substrate showing an average transmittance of 75% or more within the range of 350 nm to 390 nm can be used. In another examples, the average transmittance may be within a range of 77% or more, 79% or more, 81% or more, or 83% or more and/or 98% or less, 96% or less, 94% or less, 92% or less, 90% or less, 88% or less, 86% or less, or 84% or less.

As for the infrared absorption substrate, a substrate showing a maximum transmittance of 80% or more within the range of 350 nm to 390 nm can be used. In another examples, the maximum transmittance may be within a range of 82% or more, 84% or more, 86% or more, or 87% or more and/or 100% or less, 98% or less, 96% or less, 94% or less, 92% or less, 90% or less, or 88% or less.

As for the infrared absorption substrate, a substrate having a transmittance in the range of 10% to 45% at a wavelength of 700 nm can be used. In another examples, the transmittance may be about 43% or less, 41% or less, 39% or less, 37% or less, 35% or less, 33% or less, 31% or less, or 29% or less or 12% or more, 14% or more, 16% or more, 18% or more, 20% or more, 22% or more, 24% or more, 26% or more, or 28% or more.

As for the infrared absorption substrate, a substrate exhibiting an average transmittance within a range of 5% to 30% and within a wavelength range of 700 nm to 800 nm can be used. In another examples, the average transmittance may be within a range of 7% or more, 9% or more, 11% or more, 13% or more, 15% or more, 15.5% or more, 16% or more, or 16.5% or more and/or 28% or less, 26% or less, 24% or less, 22% or less, 20% or less, 18% or less, or 17% or less.

As for the infrared absorption substrate, a substrate exhibiting a maximum transmittance within a range of 10% to 45% and within a wavelength range of 700 nm to 800 nm can be used. In another examples, the maximum transmittance may be within a range of 12% or more, 14% or more, 16% or more, 18% or more, 20% or more, 22% or more, 24% or more, 26% or more, or 28% or more and/or 43% or less, 41% or less, 39% or less, 37% or less, 35% or less, 33% or less, 31% or less, or 29% or less.

As for the infrared absorption substrate, a substrate exhibiting an average transmittance in a range of 3% to 20% and within a wavelength range of 800 nm to 1,000 nm can be used. In another examples, the average transmittance may be further adjusted within a range of 5% or more, 7% or more, 9% or more, or 11% or more and/or within a range of 18% or less, 16% or less, 14% or less, or 12% or less.

As for the infrared absorption substrate, a substrate exhibiting a maximum transmittance in a range of 5% to 30% and within a wavelength range of 800 nm to 1,000 nm can be used. In another examples, the maximum transmittance may be within a range of 7% or more, 9% or more, 11% or more, 13% or more, or 15% or more and/or 28% or less, 26% or less, 24% or less, 22% or less, 20% or less, 18% or less, or 16% or less.

As for the infrared absorption substrate, a substrate exhibiting an average transmittance in a range of 10% to 50% within a wavelength range of 1,000 nm to 1,200 nm can be used. In another examples, the average transmittance may be further adjusted within a range of 12% or more, 14% or more, 16% or more, 18% or more, 20% or more, 22% or more, 24% or more, or 25% or more and/or 48% or less, 46% or less, 44% or less, 42% or less, 40% or less, 38% or less, 36% or less, 34% or less, 32% or less, 30% or less, 28% or less, or 26% or less.

As for the infrared absorption substrate, a substrate exhibiting a maximum transmittance in a range of 10% to 70% within a wavelength range of 1,000 nm to 1,200 nm may have a transmission band. In another examples, the maximum transmittance may be 12% or more, 14% or more, 16% or more, 18% or more, 20% or more, 22% or more, 24% or more, 26% or more, 28% or more, 30% or more, 32% or more, 34% or more, or 36% or more and/or 68% or less, 66% or less, 64% or less, 62% or less, 60% or less, 58% or less, 56% or less, 54% or less, 52% or less, 50% or less, 48% or less, 46% or less, 44% or less, 42% or less, 40% or less, 38% or less, or 37% or less.

The infrared absorption substrate can be combined with the absorption membrane to form a desired optical filter. As such a substrate, a substrate known as a so-called infrared absorbing glass can be used. Such glass is an absorption type glass manufactured by adding CuO or similar composition to a fluorophosphate-based glass or a phosphate-based glass.

Accordingly, in an embodiment of the present specification, a CuO-containing fluorophosphate glass substrate or a CuO-containing phosphate glass substrate may be used as the infrared absorption substrate. The phosphate glass also includes K phosphate glass where a portion of the glass structure is composed of SiO₂. As such absorption-type glass is publicly known, the glass disclosed in Korean Patent No. 10-2056613 or other commercially available absorption-type glass (e.g., commercially available products from Hoya, Schott, PTOT, etc.), for example, can be used.

Such an infrared absorption substrate contains copper. In the present specification, a substrate having a copper content within the range of 1% by weight to 7% by weight may be used. In other examples, the copper content may be about 1.5% by weight or more, 2% by weight or more, 2.5% by weight or more, 2.6% by weight or more, 2.7% by weight or more, or 2.8% by weight or more or 6.5% by weight or less, 6% by weight or less, 5.5% by weight or less. It may be less than 5% by weight, less than 5% by weight, less than 4.5% by weight, less than 4% by weight, less than 3.5% by weight, less than 3% by weight, or less than 2.9% by weight. A substrate having such the copper content is likely to exhibit the above-described optical properties and can be combined with the resin film to form an optical filter with desired properties.

The copper content can be confirmed by using X-ray fluorescence analysis equipment (WD XRF, Wavelength Dispersive X-Ray Fluorescence Spectrometry). When the X-ray is irradiated to a specimen (a substrate) from the equipment, characteristic secondary X-ray is generated from respective elements of the specimen and the equipment can detect the secondary X-ray with respect to the wavelength of each element. The intensity of the secondary X-rays is proportional to the element content, and thus, quantitative analysis can be performed through the intensity of the secondary X-ray. The thickness of the infrared absorption substrate may be adjusted within a wavelength range of, for example, about 0.03 mm to 5 mm, but is not limited to.

The optical filter may include other known components required in addition to the substrate and the absorption membrane. For example, the dielectric layer may additionally include a so-called dielectric layer on one or both sides of the substrate.

FIGS. 2 and 3 are examples of optical filters where a dielectric film 300 is added and illustrate a case where the dielectric film 300 is formed on one or both sides of a stacked structure including a substrate 100 and a resin film 200.

Such the dielectric film is a film composed of repeatedly stacking a dielectric material with a low refractive index and a dielectric material with a high refractive index. It is also used to form a so-called IR reflection layer and an anti-reflection (AR) layer. In the present specification, such a known IR reflection layer or a dielectric film for forming the AR layer may be applied. Accordingly, the dielectric film may have a multi-layer structure including at least two types of sub-layers each having a different refractive index and may include a multi-layer structure where the two types of sub-layers are repeatedly stacked.

The material forming the dielectric film, in other words, the material forming each sub-layer is not particularly limited and publicly known material can be applied. In general, SiO₂ or fluorides such as Na₅Al₃Fl₄, Na₃AlF₆, or MgF₂ may be used to manufacture low-refractive sub-layers and amorphous silicon, TiO₂, Ta₂O₅, Nb₂O₅, ZnS, or ZnSe, etc. may be used to manufacture high-refractive sub-layers, but the material applied in the present specification is not limited to.

In one example, the dielectric film included in the optical filter may have a shortest wavelength of 710 nm or longer that exhibits a reflectance of 50% within a wavelength range of 600 nm to 900 nm or such the wavelength may not exist. Additionally, when the above wavelength does not exist, the maximum reflectance of the dielectric film is less than 50% in the wavelength range of 600 nm to 900 nm. The shortest wavelength that exhibits a reflectance of 50%, if present, may be, in another examples, 715 nm or longer, 720 nm or longer, 725 nm or longer, 730 nm or longer, 735 nm or longer, 740 nm or longer, 745 nm or longer, 750 nm or longer, or 754 nm or longer or 900 nm or shorter, 850 nm or shorter, 800 nm or shorter, 790 nm or shorter, 780 nm or shorter, 770 nm or shorter, or 760 nm or shorter. The shortest wavelength representing the reflectance of 50% may be within the range of the lower limit and upper limit of any of the lower limits described above, and in this case, the upper limit may be 900 nm.

By controlling the reflection characteristics of the dielectric film as described above, so-called petal flare phenomenon can be prevented. The “petal flare phenomenon” refers to a phenomenon where a red line that is not observed with the naked eyes appears in a photograph when photographing a luminous object, etc., and thus, it is called as a petal flare because the red line often takes on a shape like a flower petal based on the luminous object. As the sensitivity of the sensor included in an image capturing device is increased and the transmittance of optical filters is increased to obtain clearer pictures, the frequency of the petal flares is also increased.

Repeated reflection of near-infrared light within an image capturing device equipped with an optical filter can be considered as one of the reasons causing the petal flare phenomenon. Among the dielectric films usually formed in the optical filters, because especially the so-called IR film is formed to block light in the near-infrared region by reflection, the shortest wavelength where the dielectric film exhibits a reflectance of 50% is formed near visible light and it is usually less than 710 nm. However, reflection of near-infrared light is accelerated within the image capturing device by this dielectric film, and thus, the petal flare phenomenon occurs.

In the present invention, however, infrared light can be effectively blocked even when the shortest wavelength where the dielectric film exhibits a reflectance of 50% is adjusted to 710 nm or longer, and thus, the petal flare phenomenon can also be prevented. The design method itself for controlling the reflection characteristics of the dielectric film is publicly known.

The optical filter may further include an absorption membrane that exhibits absorption characteristics for ultraviolet rays (referred to as an “ultraviolet absorption membrane”) as an absorption membrane that is distinct from the absorption membrane. However, this absorption membrane is not an essential component, and for example, the ultraviolet absorbent described later may be introduced into one absorption membrane together with the absorbent of Formula 1 above.

In one example, the ultraviolet absorption membrane may be designed to exhibit an absorption maximum in a wavelength range of about 300 nm to 390 nm. The ultraviolet absorption membrane may contain only a ultraviolet absorbent, or, if necessary, may include two or more types of ultraviolet absorbents.

For example, as the ultraviolet absorbent, a known absorbent that exhibits an absorption maximum in the wavelength range of about 300 nm to 390 nm can be used. Examples of which may include ABS 407 from Exiton; UV381A, UV381B, UV382A, UV386A, VIS404A from QCR Solutions Corp; HW Sands' ADA1225, ADA3209, ADA3216, ADA3217, ADA3218, ADA3230, ADA5205, ADA3217, ADA2055, ADA6798, ADA3102, ADA3204, ADA3210, ADA2041, ADA3201, ADA3202, ADA3219, ADA3225, ADA3232, ADA4160, ADA5278, ADA5762, ADA6826, ADA7226, ADA4634, ADA3213, ADA3227, ADA5922, ADA5950, ADA6752, ADA7130, ADA8212, ADA2984, ADA2999, ADA3220, ADA3228, ADA3235, ADA3240, ADA3211, ADA3221, 5220,ADA7158; and CRYSTALYN's DLS 381B, DLS 381C, DLS 382A, DLS 386A, DLS 404A, DLS 405A, DLS 405C, DLS 403A, etc., but are not limited to.

The materials and configuration methods used to construct this ultraviolet absorption membrane are not particularly limited, and known materials and configuration methods can be applied. Typically, an ultraviolet absorption membrane is formed using a material mixed with a transparent resin and an ultraviolet absorber capable of exhibiting the desired absorption maximum. At this time, the transparent resin may be a resin component applied to the absorbent composition. In addition to the layers described above, the optical filters may be added to the extent that various necessary layers do not impair the desired effect.

In the present specification, an image capturing device including the optical filter is also disclosed. At this time, the configuration of the image capturing device or the application method of the optical filter is not particularly limited, and known configurations and application methods may be applied. In addition, the use of the optical filter is not limited to the image capturing device, and can be applied to various other applications that require near-infrared ray cutting (for example, display devices such as PDPs, etc.).

The present specification also discloses an infrared sensor including the absorption membrane. The configuration of the infrared sensor is not particularly limited as long as the absorption membrane is included. For example, it can be configured by introducing the absorption membrane of the present invention into a known motion sensor, proximity sensor, or gesture sensor.

The application of the absorbent composition or the absorption membrane is not limited to the optical filter, infrared sensor, and/or image capturing device. The absorbent composition or the absorption membrane can be applied to various other applications that require infrared cutting (for example, display devices such as PDP, etc.).

The absorbents will be described in detail through examples below, but the scope of the absorbents is not limited by the examples below.

### 1. Absorption Maximum Measurement Method

The maximum absorption was evaluated by a conventional method. Specifically, the sample was dissolved in chloroform solvent at a concentration of about 10⁻⁵ M and then evaluated using measuring equipment (Agilent, Varian Cary 4000).

### 2. Evaluation of Transmittance Spectrum

The transmittance spectrum was measured using a spectrophotometer (Manufacturer: Perkinelmer, Product Name: Lambda 750 Spectrophotometer) on a specimen obtained by cutting the measurement object (e.g., an absorption membrane) so that the width and height were 10 mm and 10 mm, respectively. The transmittance spectrum was measured for each wavelength according to the equipment manual. The specimen was placed on a straight line between a measuring beam of the spectrophotometer and a detector and the transmittance spectrum was confirmed by setting the incident angle of the measuring beam to 0°. The incident angle of 0° is a direction substantially parallel to the surface normal direction of the specimen. The average transmittance within a certain wavelength range in the transmittance spectrum is the result of measuring the transmittance at each wavelength while increasing the wavelength by 1 nm starting from the shortest wavelength in the wavelength range, and then calculating the arithmetic average of the measured transmittances. The maximum transmittance is the maximum transmittance among the transmittances measured while increasing the wavelength by 1 nm and the minimum transmittance is the minimum transmittance among the transmittances measured while increasing the wavelength by 1 nm. For example, the average transmittance within the wavelength range of 350 nm to 360 nm is the arithmetic mean of the transmittance measured at the wavelength of 350 nm, 351 nm, 352 nm, 353 nm, 354 nm, 355 nm, 356 nm, 357 nm, 358 nm, 359 nm, and 360 nm. The maximum transmittance within the wavelength range of 350 nm to 360 nm is the highest transmittance measured at wavelengths of 350 nm, 351 nm, 352 nm, 353 nm, 354 nm, 355 nm, 356 nm, 357 nm, 358 nm, 359 nm and 360 nm and the minimum transmittance within the wavelength range of 350 nm to 360 nm is the lowest transmittance among the transmittances measured at wavelengths of 350 nm, 351 nm, 352 nm, 353 nm, 354 nm, 355 nm, 356 nm, 357 nm, 358 nm, 359 nm, and 360 nm.

### 3. Solubility Measurement Method

The solubility of the absorbent was evaluated. Solubility was determined based on the following criteria by evaluating the solubility of the absorbent in the solvent (MC, Methylene Chloride) at room temperature (about 25° C.).

### <Solubility Judgment Criteria>

A: When solubility is 1 mass % or more
B: When the solubility is 0.5 mass % or more and less than 1 mass %
C: When the solubility is 0.2 mass % or more and less than 0.5 mass %
D: When solubility is less than 0.2 mass %

### 4. Pyrolysis Temperature (Td 5%) Analysis

Thermogravimetric Analysis (TGA) of the chemical compound was performed by using Scinco's TGA N-1000 equipment. The analysis was performed by using approximately 3 mg of sample (chemical compound) and the analysis was conducted under conditions of a temperature range of 25° C. to 800° C., a temperature increase rate of 10° C./min, and nitrogen (N₂) atmosphere of 60 cm³/min. The value at 95% weight loss (Td 5%) was used as the Td decomposition temperature.

### 5. Mass Analysis (LC-Mass)

Mass analysis of the synthesized chemical compounds was performed using a liquid chromatograph/mass spectrometer (manufactured by Thermo Fisher Scientific).

### Embodiment 1

Ionic Compound (A1) containing a cation of the Formula A and an anion of the Formula B was synthesized according to Chemical Reaction Formula 1 below.

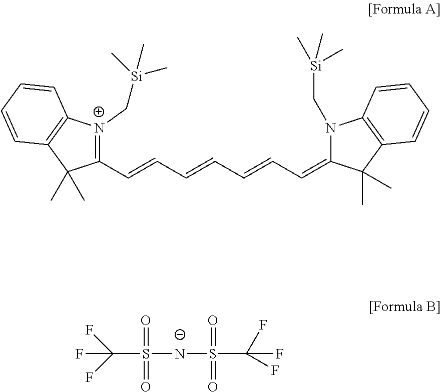 [SMILES: CC1(C)C(/C=C/C=C/C=C/C=C2\N(C[Si](C)(C)C)c3ccccc3C2(C)C)=[N+](C[Si](C)(C)C)c2ccccc21 ; O=S(=O)([N-]S(=O)(=O)C(F)(F)F)C(F)(F)F]

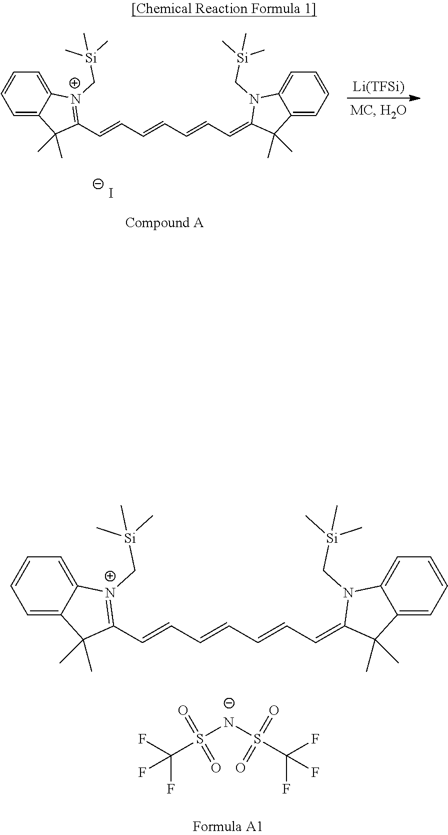

1.1 g (1.62 mmol) of Compound A in Chemical Reaction Formula 1 and 0.56 g (1.94 mmol) of lithium bis(trifluoromethanesulfonyl)imide were dissolved in 30 mL of dichloromethane, and then, additional 30 mL of water was added. The reaction was carried out at room temperature (about 25° C.) for about 2 hours. After reaction, the dichloromethane layer and the water layer were separated by an extractor, and then, it was concentrated. 100 mL of ethanol was added, and filtered under reduced pressure to obtain the target chemical (Ionic Compound A1) (0.4 g, 29.7%) (LC-MS(+) m/z 553.8, LC-MS(−) m/z 279.9).

### Embodiment 2

Ionic Compound (A2) containing a cation of the Formula C and an anion of the Formula B was synthesized according to Chemical Reaction Formula 2 below.

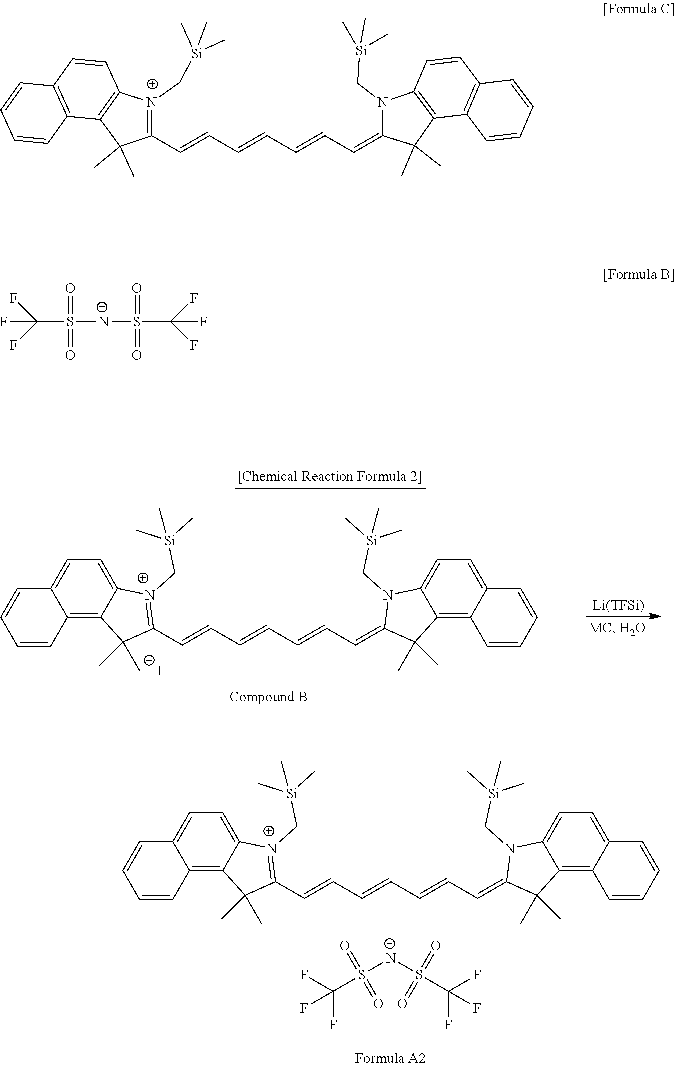

1.0 g (1.28 mmol) of Compound B of Chemical Reaction Formula 2 and 0.56 g (1.94 mmol) of lithium bis(trifluoromethanesulfonyl)imide were dissolved in 30 mL of dichloromethane, and then, additional 30 mL of water was added. The reaction was performed at room temperature (about 25° C.) for about 2 hours. After reaction, the dichloromethane layer and the water layer were separated by an extractor, and then, it was concentrated. 100 mL of ethanol was added, and filtered under reduced pressure to obtain the target chemical (Ionic Compound A2) (0.5 g, 41.8%) (LC-MS(+) m/z 653.8, LC-MS(−) m/z 280.1).

### Embodiment 3

Ionic Compound (A3) containing a cation of the Formula D and an anion of the Formula B was synthesized according to Chemical Reaction Formula 3 below.

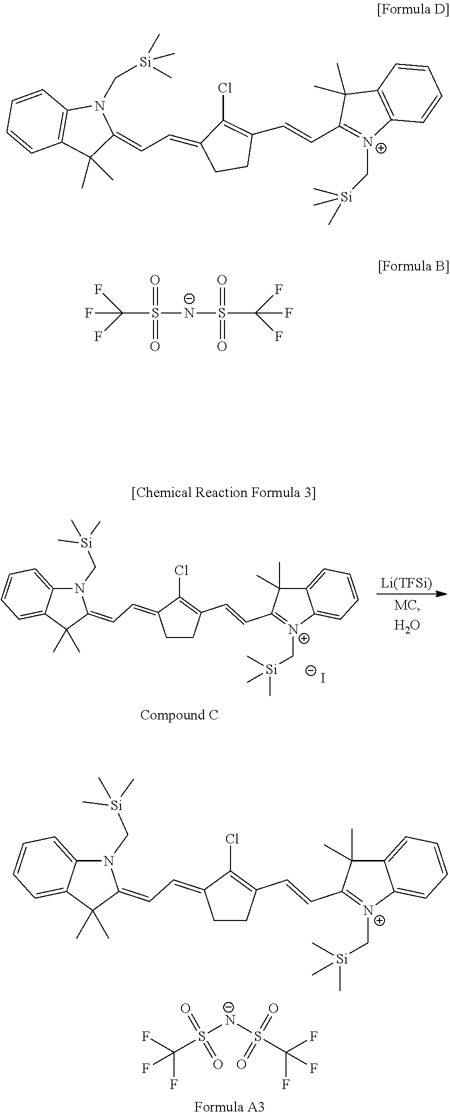

1.1 g (1.48 mmol) of Compound C of Chemical Reaction Formula 3 and 0.56 g (1.94 mmol) of lithium bis(trifluoromethanesulfonyl)imide were dissolved in 30 mL of dichloromethane, and then, additional 30 mL of water was added. The reaction was carried out at room temperature (about 25° C.) for about 2 hours. After reaction, the dichloromethane layer and the water layer were separated by an extractor, and then, it was concentrated. 100 mL of ethanol was added, and filtered under reduced pressure to obtain the target chemical (Ionic Compound A3) (0.3 g, 22.6%) (LC-MS(+) m/z 614.1, LC-MS(−) m/z 280.1).

### Comparative Example 1

Ionic Compound (A4) containing a cation of the Formula E and an anion of the Formula B was synthesized according to Chemical Reaction Formula 4 below.

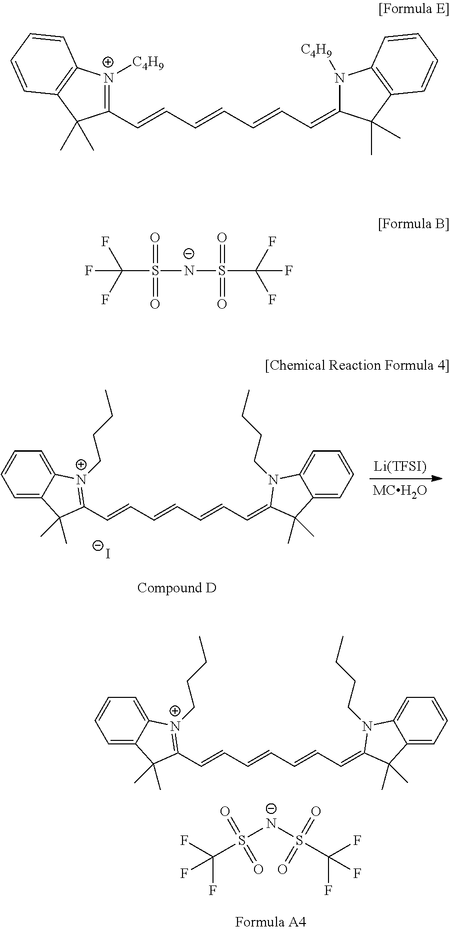

2.1 g (3.38 mmol) of Compound D of Chemical Reaction Formula 4 and 0.56 g (1.94 mmol) of lithium bis(trifluoromethanesulfonyl)imide were dissolved in 30 mL, of dichloromethane, and then, additional 30 mL, of water was added. The reaction was performed at room temperature (about 25° C.) for about 2 hours. After the reaction, the dichloromethane layer and the water layer were separated by an extractor, and then, it was concentrated. 100 mL, of ethanol was added, and filtered under reduced pressure to obtain the target chemical (Ionic Compound A4) (1.1 g, 42.0%) (LC-MS(+) m/z 493.4, LC-MS (−) m/z 280.0).

### Comparative Example 2

Ionic Compound (AM) containing a cation of the Formula F and an anion of the Formula B was synthesized according to Chemical Reaction Formula 5 below.

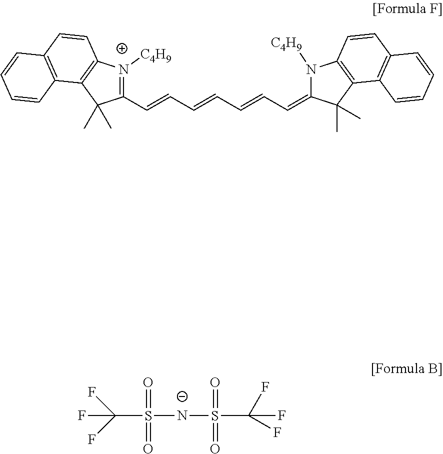

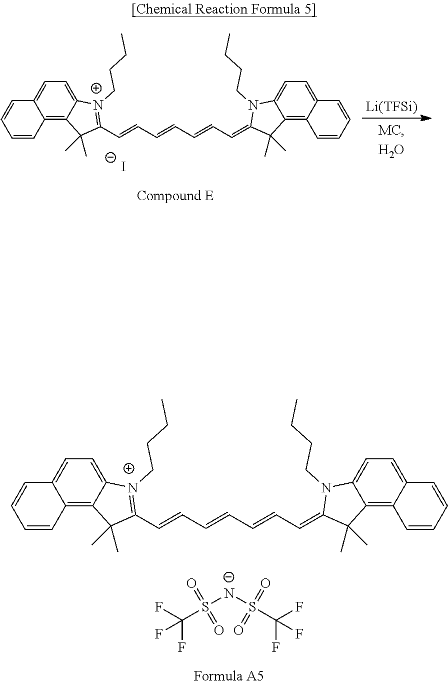

2.5 g (3.47 mmol) of Compound E of Chemical Reaction Formula 5 and 0.56 g (1.94 mmol) of lithium bis(trifluoromethanesulfonyl)imide were dissolved in 30 mL, of dichloromethane, and then, additional 30 mL, of water was added. The reaction was performed at room temperature (about 25° C.) for about 2 hours. After the reaction, the dichloromethane layer and the water layer were separated by an extractor, and then, it was concentrated. 100 mL, of ethanol was added, and filtered under reduced pressure to obtain the target chemical (Ionic Compound A5) (1.5 g, 49.5%) (LC-MS(+) m/z 593.5, LC-MS(−) m/z 280.0).

### Comparative Example 3

Ionic Compound (A6) containing a cation of the Formula F and an anion of the Formula B was synthesized according to Chemical Reaction Formula 6 below.

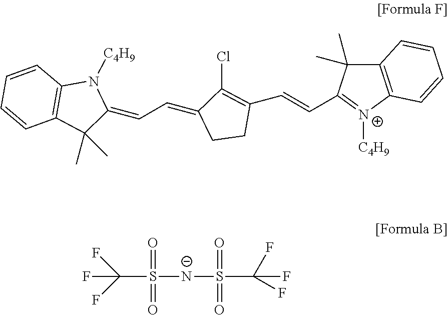

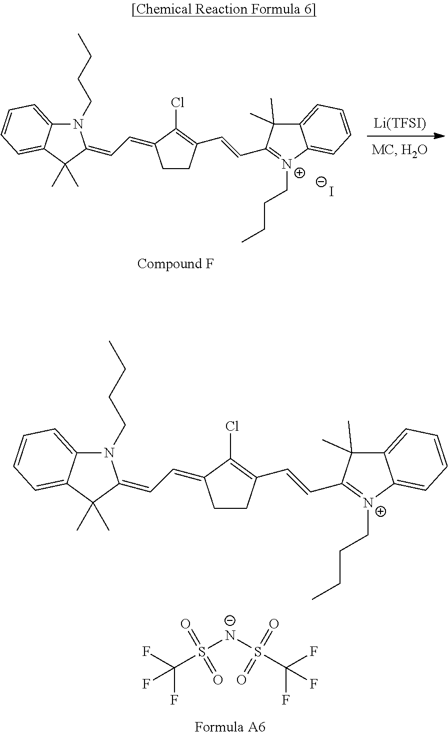 [SMILES: CCCCN1/C(=C\C=C2/CCC(/C=C/C3=[N+](CCCC)c4ccccc4C3(C)C)=C2Cl)C(C)(C)c2ccccc21 ; [I-] ; "Li(TFSI)" ; CCCCN1/C(=C\C=C2/CCC(/C=C/C3=[N+](CCCC)c4ccccc4C3(C)C)=C2Cl)C(C)(C)c2ccccc21 ; O=S(=O)([N-]S(=O)(=O)C(F)(F)F)C(F)(F)F]

1.8 g (2.64 mmol) of Compound F of Chemical Reaction Formula 6 and 0.56 g (1.94 mmol) of lithium bis(trifluoromethanesulfonyl)imide were dissolved in 30 mL of dichloromethane, and then, additional 30 mL of water was added. The reaction was performed at room temperature (about 25° C.) for about 2 hours. After the reaction, the dichloromethane layer and the water layer were separated by an extractor, and then, it was concentrated. 100 mL of ethanol was added, and filtered under reduced pressure to obtain the target chemical (Ionic Compound A6) (0.9 g, 40.8%) (LC-MS(+) mu/z 553.7, LC-MS(−) m/z 280.0).

Table 1 below summarizes the properties of the absorbents of Embodiments 1 to 3 and Comparative Examples 1 to 6. In Table 1, Td 5% means 5% of pyrolysis temperature.

TABLE 1

| | Maximum Absorption | Td 5% | |
|---|---|---|---|
| | Wavelength (nm) | (° C.) | Solubility |
| Embodiment 1 | 763 | 231 | A |
| Embodiment 2 | 803 | 230 | A |
| Embodiment 3 | 817 | 216 | A |
| Comparative Example 1 | 761 | 215 | A |
| Comparative Example 2 | 801 | 211 | A |
| Comparative Example 3 | 815 | 186 | A |

### Test Example 1

A coating solution was prepared by mixing silicone resin (Dow Chemical, RSN-0217), absorbent, and solvent (cyclohexanone) as resin components. The mixing ratio of the resin component, absorbent, and solvent was set to be 69.3:0.99:29.7 in weight ratio (resin:absorbent:solvent).

The coating solution was coated on a transparent substrate (a glass substrate manufactured by SCHOTT) and maintained at 140° C. for about 2 hours to form an absorption membrane with a thickness of about 6 μm. In the above, the absorbents of Embodiment 1, 2, and 3 and Comparative Examples 1, 2, or 3 were used as the absorbent.

Tables 2 and 3 below summarize the transmittance of the absorption membrane before and after reliability evaluation in the ultraviolet and near-infrared regions. The reliability evaluation is an evaluation of maintaining the absorption membrane at 85° C. and under 85% relative humidity condition for 120 hours. In Table 2, B refers to the result before performing the reliability evaluation and A refers to the result after performing the reliability evaluation. Additionally, in Table 2, λₘₐₓ means the transmittance at the absorption maximum.

In Tables 2 and 3, Δ is the rate of change (%) of each characteristic before and after the reliability evaluation, calculated as 100×(AB)/B, where Δ is the value indicated by A in Table 2, B is the value indicated by B in Table 2. In Table 2, Tₘₐₓ refers to the highest transmittance within the corresponding wavelength region, Tₐᵥₑ refers to the average transmittance within the corresponding wavelength region, and Tₘᵢₙ refers to the minimum transmittance within the corresponding wavelength region.

TABLE 2

| | | Example 1 | | | Example 2 | | | Example 3 | | |
|---|---|---|---|---|---|---|---|---|---|---|
| | | B | A | Δ | B | A | Δ | B | A | Δ |
| 425~465 | Tₘᵢₙ | 87.9 | 88.0 | 0.1 | 88.6 | 88.7 | 0.1 | 86.8 | 87.0 | 0.1 |
| nm | Tₐᵥₑ | 89.7 | 89.8 | 0 | 89.5 | 89.5 | 0.1 | 87.8 | 87.9 | 0.1 |
| 466~480 | Tₘᵢₙ | 90.7 | 90.7 | 0 | 89.8 | 89.8 | 0.1 | 88.1 | 88.2 | 0.1 |
| nm | Tₐᵥₑ | 91.1 | 91.1 | 0 | 90.3 | 90.3 | 0 | 89.4 | 89.5 | 0.1 |
| 480~560 | Tₐᵥₑ | 91.4 | 91.4 | 0 | 91.3 | 91.3 | 0 | 91.0 | 91.0 | 0. |
| nm | | | | | | | | | | |
| λₘₐₓ | Tₘₐₓ | 11.5 | 12.3 | 0.8 | 33.5 | 34.9 | 1.4 | 11.5 | 12.3 | 0.8 |

TABLE 3

| | | Comparative Example 1 | | | Comparative Example 2 | | | Comparative Example 3 | | |
|---|---|---|---|---|---|---|---|---|---|---|
| | | B | A | Δ | B | A | Δ | B | A | Δ |
| 425~465 | Tₘᵢₙ | 86.5 | 87.5 | 1.0 | 88.9 | 88.7 | −0.2 | 86.5 | 86.6 | 0 |
| nm | Tₐᵥₑ | 88.7 | 88.9 | 0.2 | 89.6 | 89.6 | 0 | 87.3 | 87.0 | −0.2 |
| 466~480 | Tₘᵢₙ | 89.8 | 89.1 | −0.7 | 89.8 | 89.7 | −0.1 | 88.0 | 87.5 | −0.6 |
| nm | Tₐᵥₑ | 90.1 | 89.5 | −0.6 | 90.2 | 90.2 | 0.1 | 88.9 | 88.0 | −0.9 |
| 480~560 | Tₐᵥₑ | 90.1 | 89.8 | −0.3 | 91.2 | 90.9 | −0.3 | 90.4 | 89.2 | −1.3 |
| nm | | | | | | | | | | |
| λₘₐₓ | Tₘₐₓ | 21.3 | 36.0 | 14.7 | 41.7 | 59.8 | 12.8 | 21.6 | 33.0 | 11.4 |

### Test Example 2

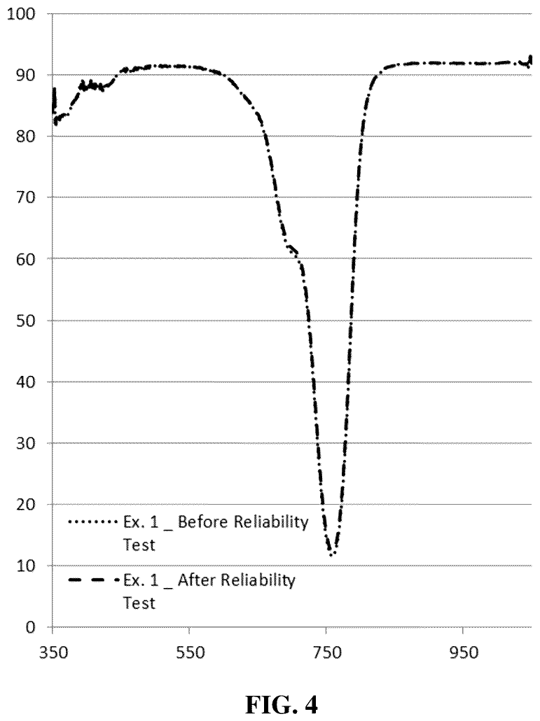

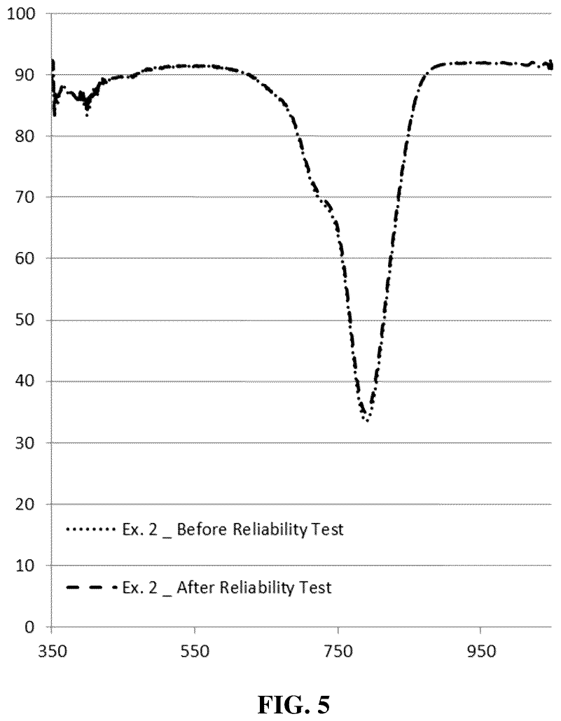

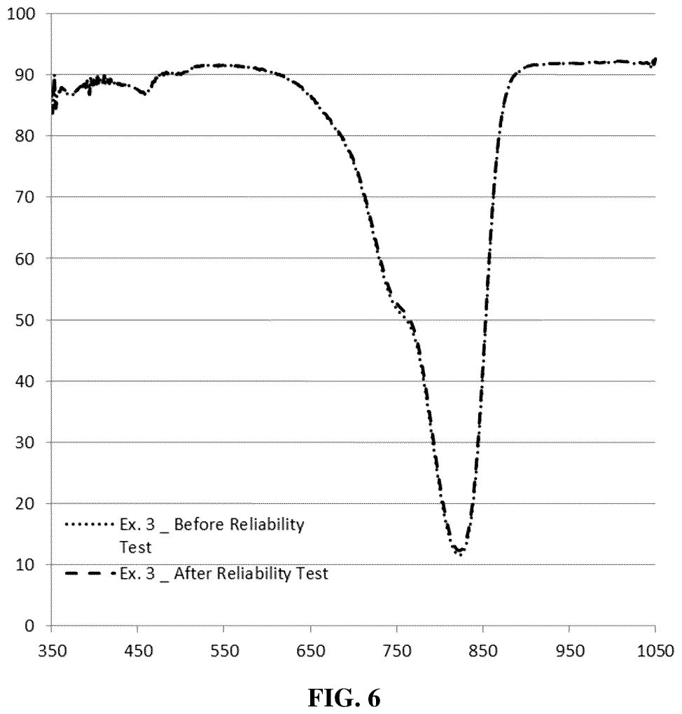

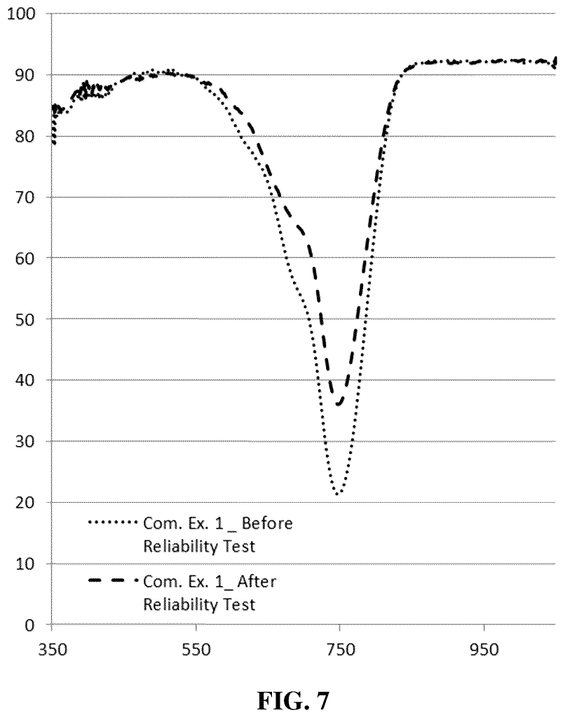

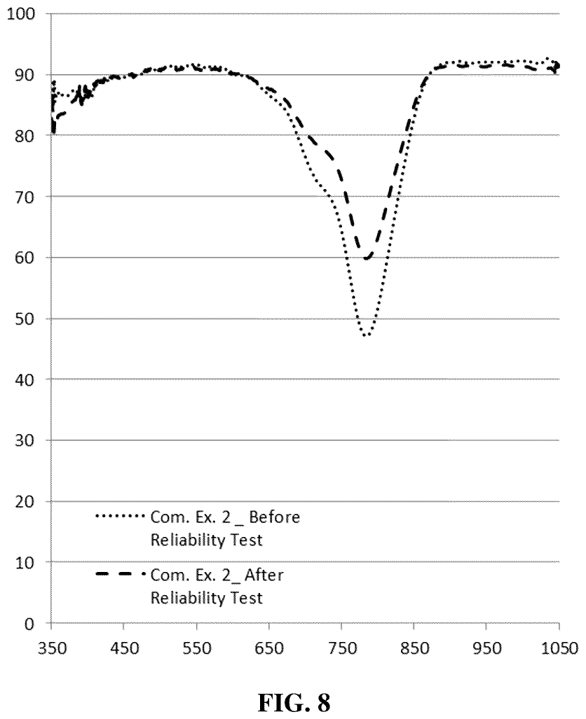

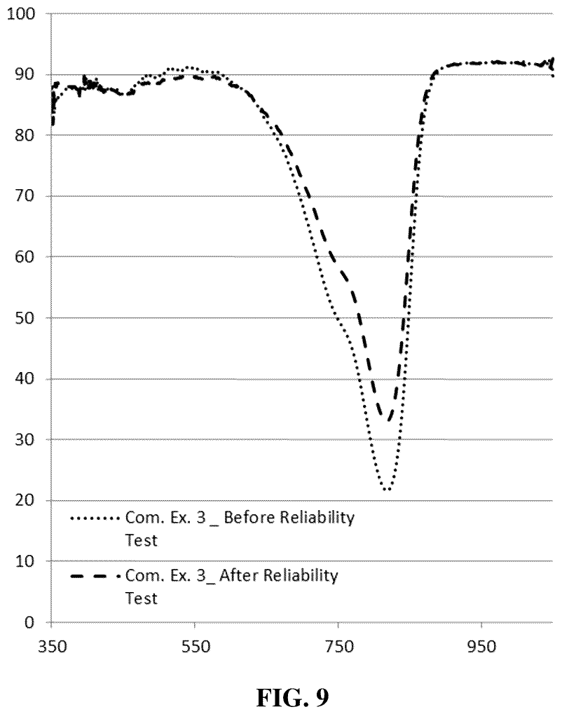

FIGS. 4 to 6 are the absorption spectra of the absorption membranes prepared in Test Example 1 using the absorbents of Embodiments 1 to 3, respectively. FIGS. 7 to 9 are the absorption spectrum of the absorption membrane prepared in Test Example using the absorbents of Comparative Examples 1 to 3, respectively. In the FIGS., the spectra indicated before reliability is the result immediately after preparing the absorption membrane, and the spectra indicated after reliability is the result after high temperature/high humidity evaluation was performed on the absorption membrane. The high temperature/high humidity evaluation is an evaluation where the absorption membrane is maintained at a temperature of 85° C. under a relative humidity of 85% for 120 hours.

From the FIGS., it can be seen that the absorption membranes using the absorbents of Embodiments 1 to 3 maintain the same light absorption characteristics before and after reliability with almost no change. When the absorbents of Comparative Examples 1 to 3 are applied, however, it can be seen that the light absorption characteristics are almost lost after high temperature/high humidity evaluation.

The main parameters for the FIGS. are summarized in Tables 4 and 5 below. In Tables 4 and 5, A_{f} is the transmittance at the absorption maximum wavelength of the absorption membrane maintained at 85° C. under 85% relative humidity for 120 hours and λ_{f} is the absorption maximum wavelength for after case. Aᵢ is the transmittance at the absorption maximum wavelength of the absorption membrane before being maintained at 85° C. under 85% relative humidity for 120 hours and λᵢ is the absorption maximum wavelength for before case.

In Tables 4 and 5, ΔA is a value calculated as 100×(A_{f}−Aᵢ)/Aᵢ, and Δλ is a value calculated as 100×(λ_{f}−λᵢ)λᵢ.

TABLE 4

| | Embodiment 1 | Embodiment 2 | Embodiment 3 |
|---|---|---|---|
| Aᵢ (%) | 11.5 | 33.5 | 11.5 |
| A_{f} (%) | 12.3 | 34.9 | 12.3 |
| ΔA (%) | 6.6 | 4.2 | 6.7 |
| λᵢ (nm) | 758 | 790 | 82. |
| λ_{f} (nm) | 758 | 790 | 823 |
| Δλ (%) | 0 | 0 | 0 |

TABLE 5

| | Comparative | Comparative | Comparative |
|---|---|---|---|
| | Example 1 | Example 2 | Example 3 |
| Aᵢ (%) | 21.3 | 47.1 | 21.6 |
| A_{f} (%) | 36.0 | 59.8 | 33.0 |
| ΔA (%) | 69.0 | 27.1 | 52.6 |
| λᵢ (nm) | 748 | 784 | 819 |
| λ_{f} (nm) | 747 | 785 | 818 |
| Δλ (%) | −13 | 13 | −12 |

Comparing the results of the FIGS. and Tables 4 and 5, the absorbent of the Embodiments and the absorbent of the Comparative Examples are similar in the spectral characteristics of the absorbent itself, but as it can be seen that a significant difference is shown in the absorption characteristics before high temperature/high humidity evaluation and the absorption characteristics before high temperature/high humidity evaluation when the absorbent was applied to the absorption membrane. From this perspective, it can be confirmed that the absorbent of the present invention has excellent compatibility with the resin component that forms the absorption membrane due to its unique structure and has excellent heat resistance, so that it can effectively form an absorption membrane with excellent performance.

1. An absorbent comprising a cation represented by Formula 1: 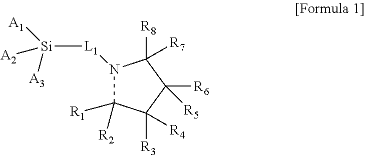
wherein
A₁ to A₃ are each independently hydrogen, halogen, an alkyl group, an alkenyl group, an alkynyl group, an alkoxy group, an aryl group, or an arylalkyl group;
L₁ is -U₁-T₁-U₂-T₂-U₃- wherein T₁ and T₂ are each independently an oxygen atom or absent and U₁ to U₃ are each independently an alkylene group, an alkylidene group, an alkenylene group, or an alkynylene group or absent;
R₁ and R₂ form an absorption edge; R₃ to R₆ or R₅ to R₈ among R₃ to R₈ form an absorption edge; and a substituent which is absence of the absorption edge among R₃ to R₈ is each independently hydrogen, halogen, a hydroxyl group, a cyano group, a nitro group, a carboxyl group, an alkyl group, an alkoxy group, an aryl group, an arylalkyl group, an alkylcarbonylamino group, an arylalkylcarbonylamino group, a haloalkylsulfonylamino group, an alkylsulfonylamino group, an arylalkylsulfonylamino group, or an amino group; and
a dotted line in Formula 1 is a single bond or a double bond wherein R₂ is absent upon the dotted line being the double bond; and
a cationic site is formed on a nitrogen atom connected to the dotted line upon the dotted line being the double bond.

2. The absorbent of claim 1, wherein A₁ to A₃ are each independently the alkyl group, the alkynyl group, the alkenyl group, or the alkoxy group in Formula 1.

3. The absorbent of claim 2, wherein the absorbent satisfies one of Conditions 1 to 3:
Condition 1: T₁, T₂, U₁ and U₃ are absent and U₂ is the alkylene group, the alkylidene group, the alkenylene group or the alkynylene group in Formula 1;
Condition 2: U₁ and U₃ are absent; one of T₁ and T₂ is oxygen and the other is absent; and U₂ is the alkylene group, the alkylidene group, the alkenylene group, or the alkynylene group in Formula 1; and
Condition 3: U₃ and T₂ are absent; T₁ is the oxygen atom, and U₁ and U₂ are each independently the alkylene group; the alkylidene group, the alkenylene group, or the alkynylene group.

4. The absorbent of claim 1, wherein the substituent which is absence of the absorption edge among R₃ to R₈ is each independently hydrogen, halogen, the hydroxy group, the cyano group, the nitro group, the carboxyl group, the alkyl group, the alkoxy group, the alkylsulfonylamino group, or the amino group.

5. The absorbent of claim 1, wherein the cation is represented by Formula 2 and Formula 3: 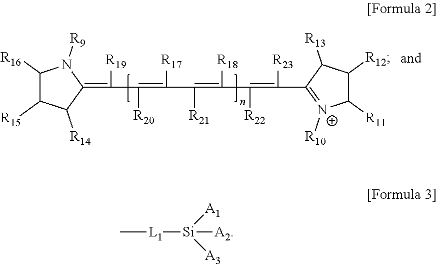
wherein
R₉ and R₁₀ are each independently hydrogen, an alkyl group, or a substituent of Formula 3 wherein at least one of R₉ and R₁₀ is the substituent of Formula 3; Ru and R₁₂ or R₁₂ and R₁₃ among R₁₁ to R₁₃ form an absorption edge together; R₁₄ and R₁₅ or R₁₅ and R₁₆ among R₁₄ to R₁₆ form an absorption edge together; a substituent which is absence of the absorption edge among R₁₁ to R₁₆ is each independently hydrogen, halogen, a hydroxyl group, a cyano group, a nitro group, a carboxyl group, an alkyl group, an alkoxy group, an aryl group, an arylalkyl group, an alkylcarbonylamino group, an arylalkylcarbonylamino group, a haloalkylsulfonylamino group, an alkylsulfonylamino group, an arylalkylsulfonylamino group, or an amino group; R₁₇ and R₁₈ are each independently hydrogen, halogen, or an alkyl group or are connected to each other to form a ring structure; R₁₉ to R₂₃ are each independently is hydrogen, halogen or an alkyl group; and n is a number greater than or equal to 1 in Formula 2; and
L₁ is -U₁-T₁-U₂-T₂-U₃- wherein T₁ and T₂ are each independently the oxygen atom or absent and U₁ to U₃ are each independently an alkylene group, an alkylidene group, an alkenylene group, an alkynylene group or absent and L₁ is connected to the nitrogen atom in Formula 2; and A₁ to A₃ are each independently hydrogen, halogen, an alkyl group, an alkenyl group, an alkynyl group, an alkoxy group, an aryl group, or an arylalkyl group in Formula 3.

6. The absorbent of claim 5, wherein R₉ and R₁₀ in Formula 2 are the substituents in Formula 3.

7. The absorbent of claim 1, wherein the absorbent exhibits an absorption maximum within a wavelength range of 600 nm to 950 nm.

8. The absorbent of claim 1, wherein a 5% thermal decomposition temperature of the absorbent is 190° C. or higher.

9. A composition comprising a resin component and the absorbent of claim 1.

10. The composition of claim 9, wherein the resin component includes at least one or more selected from a group consisting of a cyclic olefin (COP) based resin, a polyester resin, a polyarylate resin, a polysulfone resin, a polyether sulfone resin, a polyparaphenylene resin, and a polyarylene ether phosphine oxide resin, a polyimide resin, a polyetherimide resin, a polyamidoimide resin, an acrylic resin, a polycarbonate resin, a polyethylene naphthalate resin, and a silicone resin.

11. The composition of claim 9, further comprising a solvent.

12. An absorption membrane comprising a resin component and the absorbent of claim 1.

13. The absorption membrane of claim 12, wherein the resin component includes at least one or more selected from a group consisting of a cyclic olefin (COP) based resin, a polyester resin, a polyarylate resin, a polysulfone resin, a polyether sulfone resin, a polyparaphenylene resin, and a polyarylene ether phosphine oxide resin, a polyimide resin, a polyetherimide resin, a polyamidoimide resin, an acrylic resin, a polycarbonate resin, a polyethylene naphthalate resin, and silicone resin.

14. The absorption membrane of claim 12, wherein the absorption membrane exhibits an absorption maximum within a wavelength range of 600 nm to 950 nm.

15. The absorption membrane of claim 12, wherein an absolute value of ΔA in Equation 1 is 10% or less:
$$Δ A = 100 \times ( {A}_{f} - {A}_{i} ) / {A}_{i} , \qquad [ Equation 1 ]$$
wherein
A_{f} is a transmittance at an absorption maximum wavelength of the absorption membrane after the absorption membrane is maintained at 85° C. under 85% relative humidity condition for 120 hours and Aᵢ is a transmittance at an absorption maximum wavelength of the absorption membrane before the absorption membrane is maintained at 85° C. under 85% relative humidity condition for 120 hours in Equation 1; and
the absorption maximum wavelength exists within a wavelength range of 600 nm to 950 nm.

16. The absorption membrane of claim 12, wherein an absolute value of Δλ in Equation 2 is 10% or less:
$$Δ λ = 100 \times ( {λ}_{f} - {λ}_{i} ) / {λ}_{i} , \qquad [ Equation 2 ]$$
wherein
λ_{f} is an absorption maximum wavelength of the absorption membrane after the absorption membrane is maintained at 85° C. under 85% relative humidity condition for 120 hours and λᵢ is an absorption maximum wavelength of the absorption membrane before the absorption membrane is maintained at 85° C. under 85% relative humidity condition for 120 hours in Equation 2; and
the absorption maximum wavelength exists within a wavelength range of 600 nm to 950 nm.

17. An optical filter comprising a substrate and the absorption membrane of claim 12 formed on one or both sides of the substrate.

18. An image capturing device comprising the optical filter of claim 17.

19. An infrared sensor comprising the absorption membrane of claim 12.
